(19) United States
(12) Patent Application Publication
(10) Pub. No.: US 2005/0087725 A1
(43) Pub. Date: Apr. 28, 2005

(54) Thermochromic tire

(76) Inventor: Sebastina V. Kanakkanatt, Akron, OH (US)

Correspondence Address: BROUSE MCDOWELL, 388 SOUTH MAIN STREET, SUITE 500, AKRON, OH, 44311-4407, US

(21) Appl. No.: 10/352,015

(22) Filed: Jan. 27, 2003

Related U.S. Application Data
(60) Provisional application No. 60/351,922, filed on Jan. 25, 2002.

(51) Int. Cl. G01N 31/00; G01N 33/00

(52) U.S. Cl. 252/408.1

(57) ABSTRACT

Thermally color-changing dyes with high transition temperatures are disclosed herein. Using these thermochromic dyes and other ingredients paints, polymer compounds and single or multilayer patches are developed. These paints, compounds, patches, or combinations thereof are applied to tires or other substrates to give indication of heat build-up by a color change.

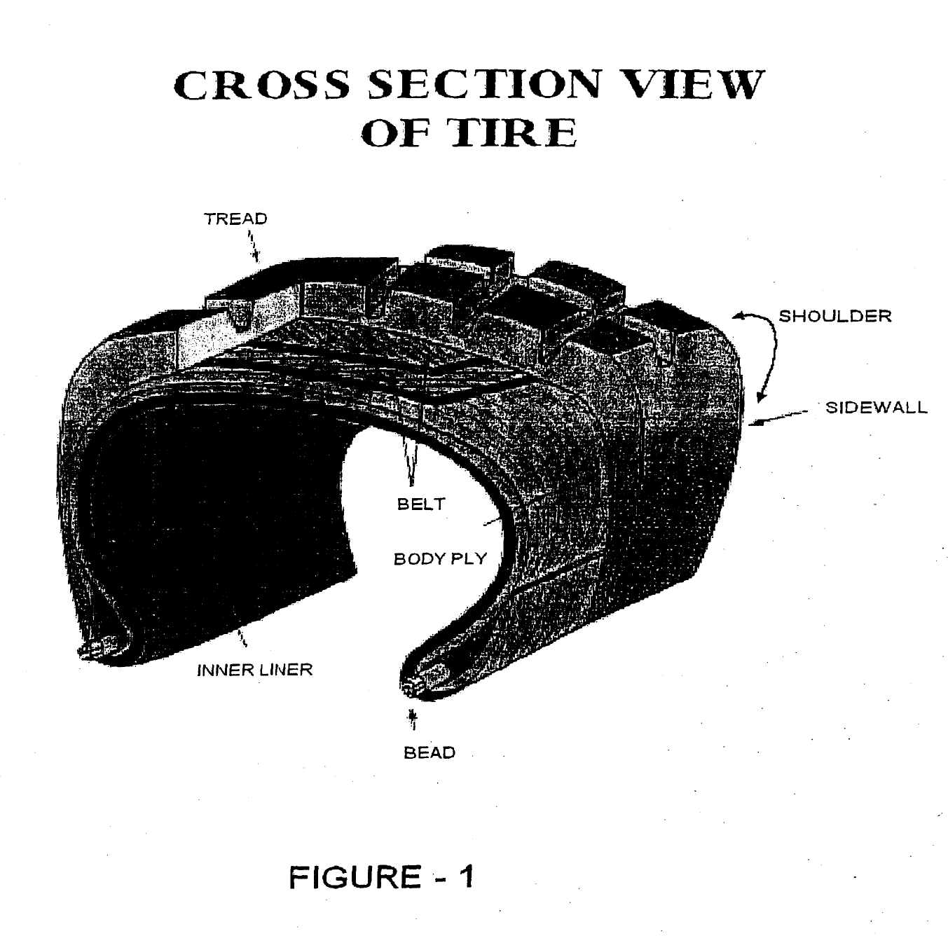

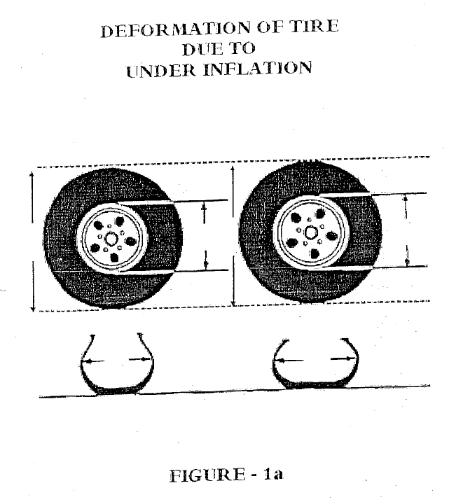

### FIG. 1

### CROSS-SECTION VIEW OF TIRE

### FIG. 1A

### DEFORMATION OF TIRE DUE TO UNDER INFLATION

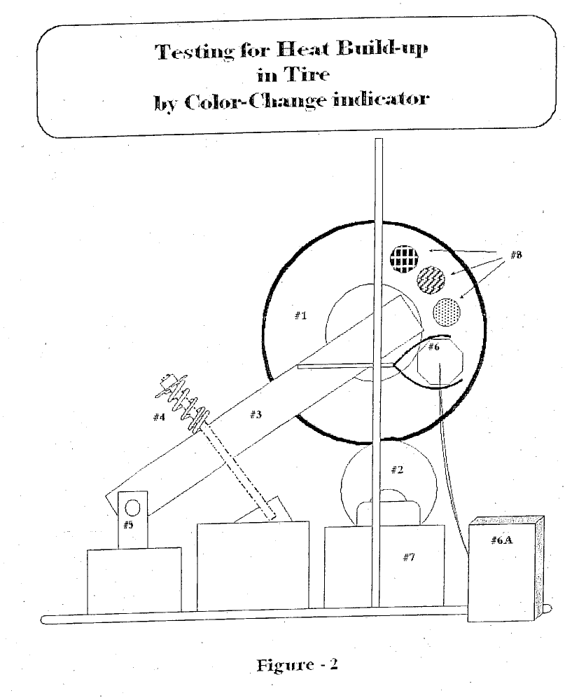

### FIG. 2

#1 A 10″ diameter tire commonly used in wheelbarrows.

#2 The rotation transfer drum.

#3 The aluminum beam (this acts as a lever of the third class in this set up).

#4 The tensioning spring (used to give the desired load on the tire).

#5 The fulcrum (the hinged lower end of the aluminum beam.)

#6 The infrared pyrometer, and

#6A The temperature display screen.

#7 A drive motor (operated at 1725 rpm in this test).

#8 Thermochromic test pieces die cut from a tensile sheet made using the recipe in Table 1.

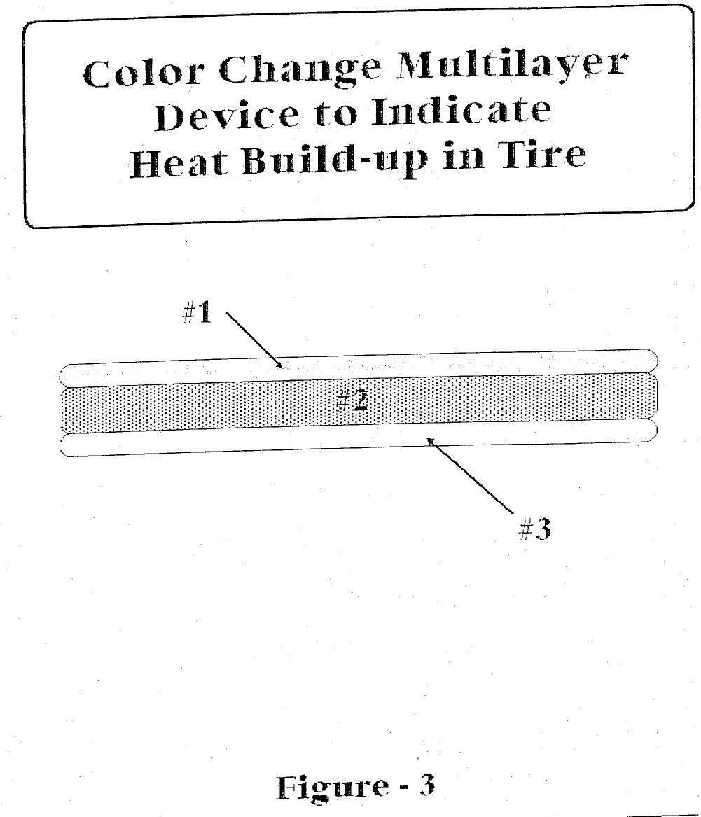

### FIG. 3

#1 The layer closest to the tire surface, a temperature barrier layer. This temperature barrier layer is used to delay the heat transfer from the tire to the active layer #2, which contains reversible thermochromic dye. This layer is especially needed when a lower temperature type thermochromic dye is used in layer #2. Alternately, this layer contains a conductive ingredient such as metallic powder or paste or metal films to transfer heat more efficiently. This is especially useful when a thermochromic dye of a high temperature type is used.

#2 This layer contains the active, reversible thermochromic dye. The thickness of this layer is varied, and optimum thickness is adjusted for the temperature type of the thermochromic dye used in this layer.

#3 This is an exterior protective layer. This is made of a transparent plastic or paint. This layer contains heat insulators, absorbers of U.V. radiation, IR radiation, antioxidants, and antiozonants.

### FIELD OF INVENTION

This invention relates to thermochromic tire or material in part thereof which reversibly changes color with temperature. More particularly, this invention relates to dyes and devices attached to tires to indicate, by a color change, heat build-up in tires by compressive deformation as the vehicle is driven. The thermochromic dye, with a special binder or substrate or combinations thereof, is incorporated on the exterior shoulder of the tire in the form of spots, patches, circular lines, or other variations thereof.

### BACKGROUND OF THE INVENTION

Tires on a vehicle are compressed at the regions of their contact on the ground due to the weight of the vehicle. As the tire rotates while the vehicle is in motion, these regions of compression shift along the circumference of the tire. Thus, any point on the tread or shoulder of the tire undergoes repetitive compression followed by relaxation once during one rotation of the tire. If the vehicle is moving at 60 miles per hour, the frequency of the repetitive compressions is about 11 Hz for a 15″ tire, and buckling at the same frequency occurs in an under-inflated tire more drastically than on a fully inflated tire (see FIG. 1). This results in a serious heat build-up causing severe damage, such as tread separation and catastrophic failure of the tire. This invention aims at giving an early warning of this potential danger by a color change visible on the surface of the tire. A paint, a stick-on patch, a rubber compound embedded in the tire, or other devices containing an appropriate temperature type thermochromic dye is an aspect of this invention. Some types of thermochromic dyes, such as those manufactured by Matsui, are commercially available, but they are not designed to detect the high temperatures used in this application unless they are modified to make them suitable for the above application. This also is an aspect of this invention. Development of a higher temperature type thermochromic dye and devices made therefrom for application on tires is another aspect of this invention.

The Concept

“Compressive deformation in under inflated tires causes heat build-up; this can be serious enough to damage the tire catastrophically. It will be a tremendous advantage if an early warning of this potential danger is given by a color change. The whitewall of the tire can be compounded with thermochromic dye of the appropriate temperature type so that a color change would occur at or below the temperature of the tire failure.” Sebastian V. Kanakkanatt, Feb. 23, 1999.

Experimental Proof of the Concept:

In order to prove this concept, a tire-testing device was built (see FIG.2). This consisted of a 10″ tire commonly used for wheelbarrows, mounted on one end of an aluminum beam with hinge (fulcrum) on the other end. Load spring is mounted at a desired point between the two ends to make it function as a lever of the third class. A rotating transfer drum is connected to a drive motor operated at 1725 rpm. The ratio of the tire diameter to that of the rotation transfer drum is such that the wheel rotates at a speed to give the compression frequency on any paint or tread or buckling frequency on any point on the shoulder is about 11 Hz. This is approximately equivalent to an operating speed of a vehicle with a 15″ tire at 60 mph.

Surface temperature is monitored using an infrared pyrometer. Interior temperatures at two select locations are measured using a dual input digital thermometer.

The tensioning spring is located at a point on the beam, to give a load arm to effect a ratio of ⅓; thus a load of 300 lb. applied by spring gives an effective load of 100 lb. on the wheel.

Die cut test pieces, 0.03″ thickness and 1″ diameter in are bonded to the surface of the tire. The tire is rotated by the drive motor operated at 1725 rpm coaxially connected to the rotation transfer drum.

The surface temperature is monitored by a (non-contact) infrared pyrometer during the operation (rotation) while the interior temperatures are observed immediately after stopping the rotation.

The rotation of the tire was stopped when the color of the test piece changed. The infrared pyrometer recorded a temperature of 62θ C. on the test piece, and 67θ C. on the tire surface. The dual input digital thermometer measured 90θ C. in the interior of the tire and 75θ C. just below the test piece:

TABLE 1

| Ingredient | Phr |
|---|---|
| Medium NBR chemigum N 608 | 100.00 |
| Sulfur | 1.50 |
| Stearic acid | 1.00 |
| Zinc oxide | 5.00 |
| Clay | 38.00 |
| Hi Sil 532 | 45.00 |
| Paraplex G 25 | 4.00 |
| MBTS (Mercaptobenzothiazole) | 1.00 |
| TMTDS (tetramethyl thiuram disulfide) | 0.25 |
| Chromicolor (PVC/plasticizer, type 60) | 110.00 |

The ingredients were mill mixed and cured at 320 F. for 15 minutes. Test sheets of 0.03″ thickness were made and test pieces of 1″ diameter were die cut. These were used in the tire-testing device. (See FIG. 2)

### SUMMARY OF THE INVENTION

In view of the above concept and application, the present inventor intends to provide novel thermochromic dyes and devices incorporating such dyes therein. These thermochromic dyes are either the negative type, in which the color fades away on temperature rise, or the positive type, ColorSine dyes manufactured by United Polymer Technology LLC in which the color appears on temperature rise. A third type, in which a color change from one to another appears, is also developed in this invention for this application. These thermochromic dyes may be microencapsulated in an appropriate cell wall material or macroencapsulated in an appropriate substrate, as in a monolithic type compound.

The devices made from the above mentioned types of thermochromic dyes might be a paint in printable or sprayable form, containing abrasion resistant ingredients in them or applied as a second coat.

These devices are also made, as disclosed in the invention, by compounding the thermochromic dyes in a rubber or plastic compound in a single layer or multilayer structure to be applied as an implant near the surface of the tire.(See FIG. 3)

Since these devices are exposed to the natural elements such as heat, cold, ultraviolet light, water, oxygen, ozone, and the like, protective coating or barrier layers will be applied to the exterior of the device to extend durability and minimize or prevent color change due to stimuli from sources other than from the heat generated inside the tire.

### PRIOR ART

The thermochromic composition in this invention is disclosed in U.S. Pat. No. 6,165,234. There are a number of other patents that disclose thermochromic dyes. These are incorporated in this patent application as references. However, none of them discloses its application as an early warning device to indicate dangerous levels of heat build-up in tires. Thus, the thermochromic compositions, devices, and their application in tires are novel materials and concepts.

### DESCRIPTION OF THE PREFERRED EMBODIMENTS

The thermochromic dyes useful in the present invention are those described in U.S. Pat. No. 6,165,234, since they are of higher temperature type having transition temperatures in the neighborhood of the melting points of waxes. Thermochromic dyes manufactured by Matsui and by other manufacturers also can be used in the devices with appropriate heat absorbers/insulators or temperature barriers.

The compression and/or buckling-induced heat build-up in tires can generate temperatures of the order of 200θ C. inside the tire, depending upon the type of rubber blend, thickness of the tread, inflation level of the tire, the speed at which the vehicle is moving, the ambient temperature, and the like. Thermocouples planted in the interior of the tire can pick up and indicate the temperature profile of the tire interior. This information is available from the tire and auto manufacturers. The temperature gradient of the tire material is also known. Thus, the exterior temperature can be taken as a good enough indicator to work backwards to discern what the interior temperature is without the use of implanted temperature indicating devices. In other words, a judiciously selected temperature type used in the device above can advantageously indicate the dangerous level of heat build-up on the interior.

One of the devices has higher temperature type thermochromic dye incorporated therein. This gives a color change indication at temperature levels such as 70θ C., 90θ C., or 100θ C. on the outside, which would mean approximately 20θ C. to 30θ C. higher temperature in the interior. This temperature differential from the point of maximum temperature in the interior to the point where the temperature indicator device is planted on the outside will vary with tire material, but it is known to a sufficient degree. Again, this temperature differential can be advantageously controlled by the selection of appropriate construction designs of the temperature-indicating device. This means thermochromic dyes of a lower temperature type can also be used by designing the device to increase the temperature differential between points of maximum interior temperature and the location of thermochromic dyes. However, the accuracy of temperature indication will be reduced?

Abrasion resistant paints incorporated therein with select thermochromic dyes can be printed or sprayed on the tire. A second transparent coat containing U.V. blocking agents, antioxidants, antiozonants, and other agents giving durability to the thermochromic paint is applied. A third transparent coating is applied as a heat shield to prevent or minimize premature color change due to extreme environmental temperatures.

A rubber compound containing thermochromic dye is cured into strips that in turn are applied to the surface of the tire or implanted in preformed cavities in the exterior of the tire. Also, these rubber compounds can be incorporated in the exterior ply during the tire-building step and in situ cured during the vulcanization step of the tire making. U.V. stabilizers, antioxidants and the like, although they can be incorporated in the rubber compound, are more efficient when they are incorporated in a transparent heat barrier layer on top of the temperature-indicating layer.

Microencapsulation of the thermochromic dye utilizes capsular material in which the active ingredient is the thermochromic dye. The capsular wall can rupture during compounding and/or heating. Wall materials and thickness that can withstand these factors are selected. The wall material is not highly transparent. Thus, the color change is influenced by the translucent cell wall material. The U.V. stabilizers, antioxidants, etc. used in the cell wall also affect the observed color change. Some of these agents can be combined with active ingredients in the cell wall.

The color changing temperature-indicating device may be located anywhere on the visible side of the tire. The preferred location is at/on the shoulder; that is, between the tread and whitewall location on the tire. This is because the perceived exterior temperature is the highest at this region of the tire and because this region is closest to the interior points of maximum temperature generation, thus minimizing the temperature differential and maximizing the accuracy. In cases where the temperature differential is 20θ C., the thermochromic dye must change color at 100θ C. to indicate the temperature in the interior has reached 120θ C., but if the temperature differential is 30θ C., the thermochromic dye used in the device need only detect 90θ C. as the transition temperature. However, increasing the temperature differential decreases the accuracy of the reading.

The devices directly applied as paint, coating, implanted patches, or line such as whitewall, etc. have the minimum added temperature differential arising from the construction design of the device. Such devices need higher temperature type thermochromic dyes, such as those in U.S. Pat. No. 6,165,234 to achieve the most accurate temperature indication.

The preferred embodiments are a positive thermochromic dye described in the U.S. Pat. No. 6,165,234 patent, manufactured by United Polymer Technology LLC under the trade name ‘ColorSine’, a negative thermochromic dye, an organo-metallic thermochromic dye, microencapsulated versions of the above, a vehicle or a substrate and materials for durability and for temperature barrier. The vehicle for paint formulation may be acrylic, styrene varnish, and the like. The substrate may be selected from thermoplastics, thermosets, elastomers, and thermoplastic elastomers. Preferred thermoplastics are polyethylene, polypropylene, impact polystyrene, polyvinyl acetate, and ABS. Preferred elastomers are natural rubber, nitrile rubber, butyl rubber, SBR, polybutadiene, and EPR. Preferred thermoplastic elastomers are di-, tri-, multi-, or radial block copolymers such as Kraton-D or Kraton-G manufactured by Shell, and others such as Santoprene manufactured by Advanced Elastomer Systems, or Engage manufactured by Dupont-Dow Elastomers.

Preferred thermosets are polyurethanes, urea-formaldehyde, melamine-formaldehyde, and the like.

U.S. Pat. No. 6,165,234 is referred to and incorporated herein by reference. These dyes are hereinafter referred to as ColorSine dyes in the following discussions, examples, and claims. Other commercially available dyes including those manufactured by Matsui are incorporated herein and referred to as negative thermochromic dyes in the following discussions, examples, and claims.

ColorSine dyes which have transition temperatures such as 73θ C., 82θ C., and 94θ C. are selected. Negative thermochromic dyes having transition temperatures of 43θ C., 53θ C., and 640 C., are selected. Conventional colors hereinafter referred to as “colors,” such as yellow, orange, and red are selected. The above selections of ColorSine dyes, negative thermochromic dyes, and colors are not the only ones that can be used or modified for this application; they are just a few of the preferred ones.

Heat absorbers which can be used with the thermochromic dyes are, but are not limited to, metallic powders. These are available commercially as powder, pastes, or polymer compounds. Metallic oxides also can be used for absorbing heat. These metallic oxides also are commercially available as powders, pastes, or polymer compounds. Metallic powder or metal oxides incorporated in negative thermochromic dyes and ColorSine dyes act as heat sinks and raise the transition temperature several degrees higher. Raising the temperature of transition by incorporating the above heat sinks by mixing or compounding the negative thermochromic dye or ColorSine dye is an aspect of this invention. This aspect is illustrated by examples and incorporated in claims.

Temperature barriers for the devices to be used to indicate the heat build-up in tires are needed when negative thermochromic dyes or ColorSine dyes of lower temperature types are used to prevent premature color change of the devices. These temperature barriers are various types of thermally insulating polymers, paints, paper, mica, silica, calcium carbonate, calcium sulfate, and the like. The preferred application of these temperature barriers is as paints or thin films of varying thickness, between the active layer in the device and the surface of the tire to which the device is applied of attached.

Combinations of heat sinks and temperature barriers can be used to extend the temperature buffer between the tire surface and the contact face of the active layer in the device.

Since these devices on the sidewall of tires must be visible from outside, they are inevitably exposed to elements of nature: heat, U.V., oxygen, ozone, water, etc. Deterioration, premature color change, and/or wear and tear will occur. In order to minimize this problem, several remedies are available, and they are incorporated in this invention.

a. Premature color change due to environmental temperature is remedied by the use of a “heat shield.” This heat shield is a paint applied as a topcoat or a film used as an exterior layer of a multilayer device. Preferred materials for this are a clear coating of acrylic or other compositions, transparent rubber, or thermoplastic or thermoset used as pre-fabricated film or generated in situ, as in the case of two-component polyurethane.

b. Deterioration due to ultraviolet light, oxygen, ozone, and water is remedied by the use of U.V. inhibitors, incorporated in the topcoat paint or the exterior layer of the polymer in the form of thin film in a multilayer device. The preferred method is to incorporate antioxidants, antiozonants, and drying agents in addition to the U.V. inhibitors in the same layer, either of the paint used as a topcoat or the film used as the exterior layer. A list of preferred chemicals that can be used as U.V. inhibitors, antioxidants, antiozonants, and drying agents that can be used in this invention is incorporated herein.

Preferred U.V. absorbers are the benzophenone type, cyanoacrylate type, salicylate type, and oxalicanilide type. Examples of the benzophenone type U.V. absorbers are:

2,4-hydroxy benzophenone

2-hydroxy-4-methoxyl benzophenone

2,2′,4,4′-tetrahydroxyl benzophenone

Examples of cyanoacrylate type U.V. absorbers are:

Ethyl-2-cyano-3,3-diphenyl acrylate

2-ethylhexyl-2-cyano-3,3-diphenyl acrylate

Examples of salicylate type U.V. absorbers are:

Phenyl salicylate

p-t-butylphenyl salicylate

p-octylphenyl salicylate

Examples of oxalic anilide type U.V. absorbers are:

Ethanediamido-N-(2-ethoxyphenyl)-N′-(4-isododecylphenyl)

Antioxidants include hindered amine types, phenol types, sulfur types, and phosphorus types.

Examples of preferred antioxidants are:

Tinuvin 620 LD and others produced by Ciba with some trade name:

2,6-di-t-butyl-4-methylphenol

dilauryl-3,3′-thiodipropionate

tris(2,4-di-t-butyl phenyl) phosphite

Preferred antiozonants are:

4,4′-thiobis(6-t-butyl-m-cresole)

2,4,6-tri-t-butyl phenol

1,4-diazabicyclo[2.2.2]octane

Infrared absorbers include compounds that have the absorption maximum at or near the infrared region of 700 to 2000 nm, and do not exhibit a large absorption in the visible region of 600 to 700 nm.

Examples of preferred infrared absorbers are:

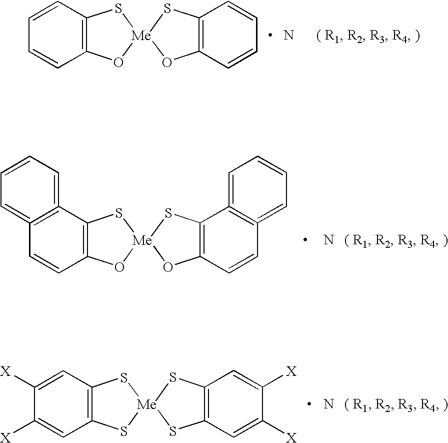

Where R is hydrogen atom, alkyl or aryl or alkoxyphenyl group.
Me is a metal atom, (nickel, palladium, or platinum.)
X is a halogen atom.

Now, examples of preferred thermochromic dyes, ColorSine dyes, paints, single layer devices, and multilayer devices are given below to illustrate details of the same. These are only examples for illustration only; they are by no means the exhaustive list of materials, paints, and devices that can be used for this application.

### EXAMPLE 1

A high temperature thermochromic dye is prepared by heating 5 parts by weight of crystal violet lactone, 15 parts by weight of Bisphenol A, 2 parts by weight of Stearamide, and 78 parts by weight of stearyl alcohol to about 130θ C. until a clear molten mass is obtained. This is cooled and used as a negative thermochromic dye. This changes color from blue to colorless at about 91θ C.

### EXAMPLE 2

A positive thermochromic dye which has a transition temperature of approximately 100θ C. is made as follows. Two parts by weight of malachite green lactone, 20 parts by weight Bisphenol A, 1 part of stearamide, 1 part by weight of KW-400 manufactured by Struktol Company, and 77 parts by weight of stearyl alcohol are heated together to about 130θ C. until a clear melted mass is obtained.

This is melted with paraffin wax at 1% by weight loading. The melt will be blue in color at around 100θ C. and white at room temperature.

### EXAMPLE 3

Thermochromic dyes previously microencapsulated and in dry powder form are used in this example. One part by weight of black thermochromic dye of temperature type 57θ C., and 99 parts by weight of polyethylene are melt mixed and pressed into films or sheets. These films or sheets will be black at the ambient temperature and white above 60θ C.

### EXAMPLE 4

The compound in example three contains an added 0.2% of a red color incorporated in it. The film made therefrom will be black at the ambient temperature, and will turn red above 600 C.

### EXAMPLE 5

The material, the thermochromic dye, ColorSine dye, with or without added color, with or without microencapsulation, one or more in combination is used in this example. This color-changing compound hereinafter is referred to as the “active.”

Five weight percent is added to 75 weight percent, an acrylic paint base known as styrene varnish which is commercially available by the same or other names or to other paint bases and stirred until a smooth paint is obtained. This is applied to a substrate such as paper, plastic film, or directly to the surface of the tire. This will change from a color to a second color on heating or when the tire reaches the temperature of transition of the device at its location.

### EXAMPLE 6

A rubber compound is made using a thermochromic dye or a ColorSine dye using the following recipe:

| | | |
|---|---|---|
| Thermochromic dye, temperature type 57 | 1.0 | phr. |
| Butyl rubber | 100.0 | phr. |
| Dicumyl peroxide | 2.5 | phr. |
| Polymerized Trimethyldihydoquinoline | 0.5 | phr. |

Sheets of varying thickness are cast and used as strips of rubber to be attached to the sidewall of the tire.

### EXAMPLE 7

Example 6 with additionally containing 1.0 phr. silver compound as follows:

| | | |
|---|---|---|
| Thermochromic dye, (temperature type 57) | 1.0 | phr. |
| Butyl rubber | 100.0 | phr. |
| Dicumyl peroxide | 2.5 | phr. |
| Silver compound | 1.0 | phr. |
| Polymerized Trimethyldihydoquinoline | 0.5 | phr. |

### EXAMPLE 8

One weight percent of black ColorSine thermochromic dye, 0.01 weight of red color supplied by French color and 98.99 weight percent of low molecular weight polyethylene are blended by heating to a temperature of 120θ C. This molten mass is cast into thin sheets or films. This will be black in color at ambient temperature; turns red at temperatures above 81θ C.

This sheet is cut into test pieces, 1″ diameter circles of 1″×½″ rectangles. These test pieces are attached to the test tire by means of an adhesive. The tire is rotated at a speed equivalent to 60 miles per hour operating speed of a vehicle giving a compressing frequency of 11 Hz. The color of the test piece changes from black to red.

### EXAMPLE 9

A coating formulation is prepared by mixing microencapsulated negative blue thermochromic dye with a transition temperature of 60θ C. and an acrylic paint base. This paint is applied by a brush on the shoulder region of the test tire. The tire is rotated at speeds as described in example 8. The blue color changes to white at about 63θ C.

### EXAMPLE 10

The test piece in example 9 is further modified using a temperature barrier layer on the inside and a heat shield layer on the outside. This piece is attached to the tire with a temperature barrier layer in contact with the tire. Another test piece without the heat barrier layer and a third test piece without the heat shield layer are also attached. A hot air blower is directed to the surface of the tire to generate a tire surface temperature of 50θ C. On rotation of the tire, the test piece without the heat barrier layer and the one without the heat shield layer change color at a temperature lower than the test piece having both a heat barrier layer and a heat shield layer. This test piece, with both a temperature barrier layer and a heat shield layer changes color at a temperature of about 100θ C. on the surface of the tire.

### EXAMPLE 11

Three weight percent of crystal violet lactone, 10 weight percent of 1,2,3-triazole, and 87 weight of sebacodihydrazide are melted together to give a homogeneous melt. This gives a thermochromic dye with a transition temperature of 100θ C. and the color changes from blue to white (clear) above 150θ C.

### EXAMPLE 12

One weight percent of the thermochromic dye, 1 weight percent of trilaurin, and 98 weight percent of polyethylene of low molecular weight of melt point of about 120θ C.-150θ C. The resulting compound is a positive thermochromic dye or ColorSine A type which will be colorless below 100θ C., burning blue above 100θ C.

1. The concept of an early warning of potential tread separation or catastrophic tire failure due to high levels of heat build-up by means of a color change on the visible side of the tire.

2. A reversible high temperature thermochromic composition which, as transition temperatures (above 70θ C.) comprising (a) an electron donating color developing organic compound selected from the group containing triaryl methane, alkyl phthalide, trialkyl indolino-benzospiropyran, alkyl and halo substituted fluoran and alkyl and amino substituted fluoran, (b) an electron accepting compound selected from a group comprising Bisphenol A, alkyl substituted phenols, hydroxy naphthalenes, halobenzotriazoles, and hydroxy benzotriazoles, (c) a medium selected from a group containing amides of fatty acids, hydrazides of fatty acids, organic polymers having a melting point in the range of 100θ C. to 250θ C. and (d) a transition temperature shifting agent selected from a group of compounds comprising polyalkyl benzaldehydes, esters of fatty acids with long chain alcohols, aryl aryl ketones, and glycerides.

3. A paint comprised of a reversible thermochromic dye having a transition temperature from 80θ C. to 200θ C., preferred range of transition temperatures being 60θ C. to 150θ C., a base consisting of compounds selected from a group comprised of acrylic polymers, thermoplastic elastomers, natural and synthetic elastomers, and the like.

4. A thermally color changing polymer compound containing a reversible thermochromic dye optionally containing a conventional color compounded as illustrated by Table 1, and cured to cut test pieces therefrom.

5. A multilayer thermochromic device as illustrated by FIG. 3, comprising a temperature barrier layer #1, an active layer #2 containing one or more reversible thermochromic dyes taken from claim 3 or from claim 4, and an exterior layer #3, containing absorbers of heat, ultraviolet light, and infrared radiation, an antioxidant, and antiozonants, optionally containing a drying agent.

6. The experimentally proven concept of attaching a device as claimed in #5 on the surface of the tire, implanted near the surface or built into the tire on an exterior ply such as the whitewall on the tire, as a circular, rectangular, or other shaped patch or as a circle on the entire sidewall of the tire or part thereof.

7. The paint in claim 3 is applied by printing, brushing, or spraying with an additional coat applied topically.

8. The topcoat in claim 7 consists of a paint base as in claim 3 and protective ingredients such as absorbers of heat, infrared, and U.V. and additionally antioxidants and antiozonants.

9. An undercoat to the paint in claim 3 is optionally applied to the tire prior to the application of paint or coatings as in claim 3.

10. The reversible thermochromic dye in claims 1 through 9 is a negative thermochromic dye.

11. The reversible thermochromic dye in claims 1 through 9 is a positive thermochromic dye.

12. The reversible thermochromic dye in claims 10 and 11 is microencapsulated.

13. The reversible thermochromic dye in claims 10 and 11 is not microencapsulated.

14. The reversible thermochromic dye contains a color to give a color change from one shade to another due to heat transferred from the tire in which heat build-up has occurred.

15. The reversible thermochromic dye is a mixture of thermochromic dyes of two or more temperature types and optionally a color, to exhibit two or more color changes, each color at a pre-determined temperature.
